(19) United States
(12) Patent Application Publication
(10) Pub. No.: US 2011/0213170 A1
(43) Pub. Date: Sep. 1, 2011

(54) ESTOLIDE DERIVATIVES USEFUL AS BIOLUBRICANTS

(76) Inventors: Daniele Vinci, Gent (BE); John Beckerdite, Lake Jackson, TX (US)

(21) Appl. No.: 13/026,581

(22) Filed: Feb. 14, 2011

Related U.S. Application Data
(60) Provisional application No. 61/308,453, filed on Feb. 26, 2010.

(51) Int. Cl. C07C 67/28; C07C 69/34

(52) U.S. Cl. 554/122

(57) ABSTRACT

A double ester composition prepared by a three-step process comprising the non-ordered steps of a homopolymerization, a transesterification, and a capping, wherein the ordered steps include a sequence of homopolymerization, capping, and transesterification, or a sequence of transesterification, homopolymerization, and capping. The ester is useful particularly as a biolubricant having a high level of renewable carbons, and may exhibit particularly desirable properties relating to pour point, thermo-oxidative stability, and viscometric behavior due to reduced or eliminated levels of unsaturation in the final double esters.

### BACKGROUND

1. Field of the Invention

The invention relates to biolubricant compositions. More particularly, the invention relates to estolide derivatives of fatty acids that have a high level of renewable raw materials and are useful as lubricants.

2. Background of the Art

The lubricants (engine and non-engine) and process fluids industries today are searching for materials that are biodegradable. Biodegradability means that the lubricants and process fluids (hereinafter “fluids”) degrade over a period of time, which may be measured by tests such as those promulgated by the Organization of Economic Co-Operation and Development (OECD), including OECD 301B and OECD 301F. Recently, interest has been increasing in fluids which are not only biodegradable, but also renewable. Renewable products contain, by definition, high levels of renewable carbons, and standards are being set to encourage increasingly greater levels of renewability. For example, the European Ecolabel now requires that hydraulic fluids must contain at least 50 percent by weight renewable carbons.

Researchers have attempted to meet requirements or recommendations for both biodegradability and renewability by including in their fluids formulations a variety of types of natural and synthesized oils. Unfortunately, many of these materials exhibit pour points that are too high to enable use in certain important applications. The pour point is the lowest temperature at which the fluid will flow, and pour points below 0 degrees Celsius (° C.), desirably below −10° C., more desirably below −15° C., and even below −25° C., are often necessary. These materials in many cases also suffer from poor thermo-oxidative stability at high temperatures (for example, above 90° C.), which may in some cases be due to the amount of unsaturation present in the acid fraction of their chemical structures.

In order to obtain these properties, research has been done on estolides. Estolides are oligomeric fatty acids which may be formed by condensation of two or more fatty acid units to yield an ester linkage. Typically this condensation is accomplished by reacting a carboxylic acid moiety onto a double bond via acid catalysis.

An example of work on estolides is disclosed in U.S. Pat. No. 6,018,063 (Isbell, et al.), which relates to esters of estolides derived from oleic acids. This patent discloses a synthesis of estolides involving homopolymerization of castor oil fatty acids or 12-hydroxystearic acid under thermal or acid catalyzed conditions.

Another example is U.S. Pat. No. 6,407,272 (Nelson, et al.), which teaches preparation of secondary alcohol esters of hydroxy acids (for example, ricinoleate esters of secondary alcohols) by reacting an ester of a hydroxy acid with a secondary alcohol in the presence of an organometallic transesterification catalyst.

Still another example is found in Patent Cooperation Treaty Publication (WO) 2008/040864, which relates to a method for synthesizing estolide esters having a specified oligomerization level and a low residual acid index. The method involves simultaneous oligomerization of a saturated hydroxy acid and esterification of the hydroxyacid by a monoalcohol.

None of the above methods, however, has been shown to produce a fully saturated material having desirable combinations of low pour point (at or below −10° C.), thermo-oxidative stability, and renewable carbons (at least 50 percent by weight). Thus, there is a need in the art for new compositions meeting these requirements, while at the same time exhibiting additional desirable or specified lubricity and viscosity properties, such that they are capable of being used in lubricant applications.

### SUMMARY OF THE INVENTION

In one embodiment the invention provides a process for preparing a double ester composition comprising the ordered steps of: (1-a) at least partially homopolymerizing a hydroxylated fatty acid or fatty ester to form a fatty acid homopolymer; (1-b) capping the fatty acid homopolymer with an acid, acid anhydride or ester to form a double ester; and (1-c) transesterifying the fatty acid homopolymer with an alcohol to form a capped fatty acid homopolymer ester; or the ordered steps of (2-a) transesterifying a hydroxylated fatty acid or fatty ester with an alcohol to form a hydroxylated fatty ester; (2-b) homopolymerizing the hydroxylated fatty ester to form a fatty acid homopolymer ester; and (2-c) capping the fatty acid homopolymer ester with an acid, acid anhydride or ester to form a double ester. The double ester compositions prepared by either of these methods represent another embodiment of the invention.

In still another embodiment the invention provides a process for preparing a double ester of a secondary hydroxy fatty acid or fatty ester, the process comprising either the ordered steps of (1-a) through (1-c), or of (2-a) through (2-c), the ordered steps being either: (1-a) partially homopolymerizing a hydroxylated fatty acid compound, using a tin-containing, titanium-containing or nitrogen-containing catalyst and removing formed alcohol, optionally by using one or more of an entrainer, reduced pressure and nitrogen sparging, to yield a product 1-X with distribution of compounds represented by Formula 1:

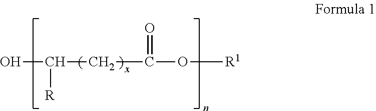

wherein in individual compounds R is an alkyl group that contains from 6 to 12 carbon atoms, R¹ is hydrogen or a methyl radical, x is an integer within a range of from 8 to 12 and n is an integer between 1 and 20, and the formed alcohol having the formula R¹OH; (1-a1) optionally recovering product 1-X from residual R¹OH and, when used, the entrainer; (1-b) reacting product 1-X with an acid that contains from 2 to 12 carbon atoms, an ester that contains from 3 to 13 carbon atoms, or an acid anhydride that contains from 4 to 24 carbon atoms, optionally using an additional amount of a tin-containing, titanium-containing or nitrogen-containing catalyst, and removing formed alcohol to yield a product 1-Y with a distribution of compounds represented by Formula 2:

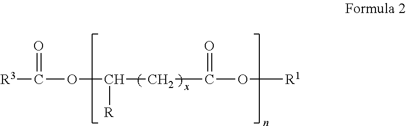

wherein R, R¹, x and n are as defined above and R³ is an alkyl group that contains from 1 to 11 carbon atoms; (1-b1) optionally recovering product 1-Y from excess acid, acid anhydride or ester added as a reactant in step (1-b); and (1-c) reacting product 1-Y with an alcohol to form product 1-Z with a distribution of compounds represented by Formula 3:

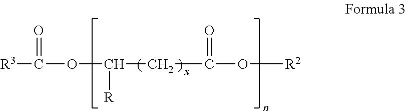

wherein R, R³, x and n are as defined above, and R² an alkyl group that contains from 1 to 20 carbon atoms; (1-c1) optionally recovering product 1-Z from alcohol and residual R¹OH added during (1-c) and acid formed during reaction of 1-Y with the acid, acid anhydride or ester added in (1-b); or the ordered steps being: (2-a) reacting a secondary hydroxyl fatty acid or fatty ester with an alcohol to form product 2-X with a distribution of compounds represented by Formula 4:

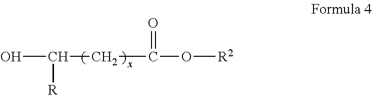

wherein R is an alkyl group that contains from 6 to 12 carbon atoms; R² is an alkyl group that contains from 1 to 20 carbon atoms, x is an integer within a range of from 8 to 12; (2-a1) optionally recovering product 2-X from residual or formed R²OH; (2-b) partially homopolymerizing product 2-X, using a tin-containing, titanium-containing or nitrogen-containing catalyst and removing the formed R²OH, optionally by using one or more of an entrainer, reduced pressure and nitrogen sparging, to yield a product 2-Y with distribution of compounds represented by Formula 5:

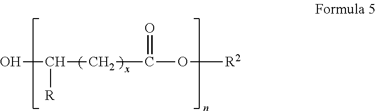

wherein in individual compounds R, R², and x are as defined above and n is an integer between 1 and 20; (2-b1) optionally recovering product 2-Y from residual R²OH and, when used, the entrainer; and (2-c) reacting product 2-Y with an acid that contains from 2 to 12 carbon atoms, an ester that contains from 3 to 13 carbon atoms, or an acid anhydride that contains from 4 to 24 carbon atoms, optionally using an additional amount of a tin-containing, titanium-containing or nitrogen-containing catalyst, to yield a product 2-Z with distribution of compounds represented by Formula 6:

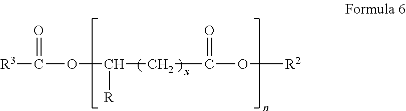

wherein R, R², R³, x and n are as defined; (2-c1) optionally recovering product 2-Y from excess acid, acid anhydride or ester added as a reactant in (2-b) and alcohol added as a reactant in (2-c).

The compounds of Formulae 1, 2, 3, 5, and 6 described above may exist in the product as a mixture or distribution of compounds which may have varying values of n. Thus, in some embodiments, average n for the distribution of compounds of Formula 1, 2, 3, 5, or 6 may be a fraction between 1.01 and 20.

### DETAILED DESCRIPTION OF THE EMBODIMENTS

The invention provides an improved process to prepare certain estolide derivatives that exhibit useful friction and wear properties, desirably low pour points, good thermo-oxidative stability, and are based on a renewable resource, such that the material may be classified as bio-based.

Preparation of the estolide derivatives may be carried out beginning with a hydroxylated fatty acid or fatty ester. In preferred embodiments this hydroxylated fatty acid or fatty acid may be, conveniently, a methyl ester of a 12-hydroxy fatty acid, such as 12-hydroxystearic acid. In general the synthesis may be via a three-step process which includes a homopolymerization, a transesterification, and a capping, but it has surprisingly been found that variation in the order of these steps, though ultimately still resulting in formation of a double ester of the starting hydroxylated material, affects the overall properties of the double ester, which is generally obtained as a mixture of final products.

In the first embodiment of the invention, the three steps are ordered as a homopolymerization, a capping, and a transesterification. In greater detail, the hydroxylated fatty acid or fatty ester is first at least partially homopolymerized to form a fatty acid or fatty ester homopolymer. This homopolymerization is desirably carried out in the presence of a tin-, titanium-, or nitrogen-containing catalyst and any forming methanol is concurrently removed. The methanol removal may be accomplished by means of an entrainer, reduced pressure, and/or nitrogen sparging. The result of this step is an oligomerized ester which includes a distribution of compounds of Formula 1, as defined hereinabove.

The oligomerized ester is then recovered from excess alcohol, residual methanol and/or the entrainer, and then capped by reacting with an acid that contains from 2 to 12 carbon atoms, an ester that contains from 3 to 13 carbon atoms, or an acid anhydride that contains from 4 to 24 carbon atoms, to form a capped estolide ester. Additional tin-, titanium-, or nitrogen-containing catalyst may optionally be employed for this capping. The distribution of product capped estolide esters may be represented by Formula 2, as defined hereinabove. The capped estolide ester may be recovered from excess acid, acid anhydride or ester.

Finally, in a transesterification step, the capped estolide ester is reacted with an alcohol having from 2 to 20 carbon atoms. In certain desirable and non-limiting embodiments, the alcohol may be selected from 2-ethylhexanol, 2-(2-butoxy-propoxy)propan-1-ol (DPnB), 1-octanol, 2-octanol, and combinations thereof. Additional tin-, titanium-, or nitrogen-containing catalyst may be employed at this point, and formed methanol is removed, yielding a double estolide ester represented by a distribution of compounds represented by Formula 3, as defined hereinabove.

In a second embodiment of the invention, the double ester composition may be prepared by a process wherein a transesterification step is first, followed by homopolymerization and, finally, capping steps. In this embodiment, (2-a) the hydroxylated fatty acid or fatty acid ester is first transesterified by reacting it with an alcohol to form product 2-X with a distribution of compounds represented by Formula 4, as defined hereinabove; (2-a1) optionally recovering product 2-X from excess alcohol; (2-b) partially homopolymerizing product 2-X, using a tin-containing, titanium-containing or nitrogen-containing catalyst and removing formed alcohol, optionally by using one or more of an entrainer, reduced pressure and nitrogen sparging, to yield a product 2-Y with a distribution of compounds represented by Formula 5 as defined hereinabove; (2-b1) optionally recovering product 2-Y from residual R²OH and, when used, the entrainer; (2-c) reacting product 2-Y with an acid that contains from 2 to 12 carbon atoms, an ester that contains from 3 to 13 carbon atoms, or an acid anhydride that contains from 4 to 24 carbon atoms, optionally using an additional amount of a tin-containing, titanium-containing or nitrogen-containing catalyst, to yield a product Z-2 with distribution of compounds represented by Formula 6, as defined hereinabove; (2-c1) optionally recovering product 2-Y from excess acid, acid anhydride or ester added as a reactant in (2-b) and alcohol added as a reactant in (2-c).

In either of the above processes, the capping step may be carried out using, in certain preferred embodiments, an acid anhydride of Formula 7:

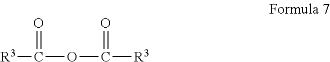

wherein R³ is as defined above with respect to Formula 2. Illustrative anhydrides include isobutyric anhydride.

In certain embodiments the double esters prepared by the inventive process are novel compositions and may exhibit a number of properties that make them useful and/or desirable for a variety of applications. These applications may include, but are not limited to, plasticizers for resins, power transmission fluids for hydraulics, heat transfer fluids, thickening agents, solvents, and surfactants. Furthermore, these compositions may also be useful in the production of polyurethanes, including foams, elastomers, coatings, and adhesives.

The double ester compositions may exhibit properties including at least one of a pour point that is less than or equal to −10° C. (measured according to ASTM D97); a viscosity index that is greater than or equal to 150; a kinematic viscosity at 40° C. that is more than 25 centistokes (cSt) (0.000025 square meters per second (m²/second)) (measured according to ASTM D445); a total acid number that is less than 1 milligram of potassium hydroxide per gram (mg KOH/g), and in particular embodiments less than 0.5 mg KOH/g; and an iodine number that is less than 3 weight percent (wt %), indicating full saturation. In particular embodiments the double esters may have a pour point that is less than −30° C., and a kinematic viscosity at 40° C. that is greater than 35 cSt (0.000035 m²/second) and preferably greater than 45 cSt (0.000045 m²/second). They may also have a hydroxyl number of less than or equal to 10, preferably less than 8, more preferably less than 5, still more preferably less than 4, and even more preferably less than 3; and an iodine number that is less than 3 weight percent (wt %), indicating full saturation. They may also exhibit desirable levels of thermo-oxidative stability (measured according to ASTM D2893), and renewable carbons (at least 50 percent by weight, measured according to ASTM D6866-08).

In carrying out the method described to prepare the capped estolide esters used in the inventive compositions, those skilled in the art should be able to easily discern suitable reaction protocols and conditions. However, it may be noted that the temperature for the homopolymerization [alternatively referred to as oligomerization or condensation] of the hydroxylated fatty acid compound, in either the first or second embodiment, and also for the azeotropic distillation of the methanol formed during the reaction, is desirably from 70° C. to 220° C., more desirably from 120° C. to 210° C., and still more desirably from 180° C. to 200° C.

The temperature for the transesterification reaction, in either the first or second embodiment, may be accomplished at a temperature from 70° C. to 220° C., and in certain particular embodiments from 120° C. to 210° C., still more particularly from 180° C. to 200° C. The branched alcohol is desirably present in an amount sufficient to provide at least one molar equivalent of alcohol for each molar equivalent of the oligomerized ester or the hydroxylated fatty acid or fatty acid ester (depending upon the embodiment).

The capping of the estolide ester is desirably carried out at a temperature from 80° C. to 160° C., more preferably from 100° C. to 140° C., and still more desirably from 110° C. to 130° C.

Optional step (1-a1), recovering product 1-X from residual methanol formed during step (1-a) and, when used, an entrainer may be accomplished via conventional procedures such as azeotropic distillation with the entrainer, preferably using an aliphatic compound having from 7 to 10 carbon atoms, most preferably 9 carbon atoms. Entrainment and removal of both residual methanol and the entrainer preferably occurs via distillation under reduced pressure (for example, 4 kilopascals (kPa)). The temperature is preferably within a range of from 100° C. to 200° C., more preferably from 120° C. to 190° C., and still more preferably from 150° C. to 180° C.

Optional step (1-b1), recovering product 1-Y from excess step (1-b) alcohol and residual methanol from step (1-a), may be accomplished via conventional procedures such as fractionated distillation. Step (1-b1) preferably involves distillation under reduced pressure (for example, 4 kPa) to effect recovery of product 1-Y. The temperature is preferably within a range of from 70° C. to 350° C., more preferably from 120° C. to 250° C., and still more preferably from 150° C. to 180° C.

Optional step (1-c1), recovering product 1-Z from excess acid, acid anhydride or ester added as a reactant in step (1-b) and acid formed during reaction of product 1-Y with the acid, acid anhydride or ester, preferably includes one or more of (1) use of reduced pressure to remove volatile materials, (2) washing one or more times with a base, such as an aqueous solution of sodium hydrogen carbonate (NaHCO₃), (3) use of absorbent materials such as magnesium silicate, activated carbon and magnesium sulfate (MgSO₄), and (4) filtration.

Numeric ranges used in this specification are inclusive of the numbers defining the range. Unless otherwise indicated, ratios, percentages, parts, and the like are by weight.

The following examples are illustrative of the invention but are not intended to limit its scope.

### EXAMPLES

### Example 1

Step 1: A glass reactor equipped with a temperature controller, overhead stirrer and Dean-Stark apparatus is charged with methyl-12-hydroxy-stearate (5296.2 grams (g)), nonane fraction (793.4 g) and tin(II)-2-ethylhexanoate (15.9 g). The mixture is then heated to 190° C. for a period of 20 hours, removing methanol by azeotropic distillation with nonane. Residual nonane fraction is distilled under reduced pressure (20 millibar (mbar), 2 kilopascals (kPa)) at 160° C., and then the reactor is cooled to 120° C.

Step 2: To the product of step 1 (463.29 g), isobutyric anhydride (93.49 g) is added. The reactor is stirred at this temperature for 2 hours. Excess anhydride and acid formed during capping are removed under reduced pressure. Temperature is then increased to 160° C. and reduced pressure is maintained for two hours, the reactor contents are then cooled to a set point temperature of 70° C., and a NaHCO₃ aqueous solution (100 milliliters (mL), 1 molar (M)) is added to the reactor with stirring. After stirring for 1 hour, water is removed under reduced pressure. Magnesium silicate (1 percent by weight (% w/w)), activated carbon (1% w/w) and MgSO₄ (1% w/w) is added to the reactor, then the material is filtered using a filter paper coated with 8 percent (%) of magnesium silicate to yield the final product.

Step 3: A Vigreux distillation column is placed between the reactor and the Dean-Stark apparatus, then 2-ethylhexanol (77.72 g) and tin(II)-2-ethylhexanoate (0.02 g) are added to the product of step 2 (357.2 g) and the mixture is heated to 190° C. for a period of 6 hours, removing methanol by fractional distillation. Excess 2-ethylhexanol is removed by distillation under pressure at 160° C. and then the reactor is cooled to 20° C. The resulting product is a light yellow liquid.

### Example 2

Step 1: A glass reactor equipped Vigreux distillation column placed between the reactor and the Dean-Stark apparatus is charged with methyl-12-hydroxy-stearate (2921.8 g), 2-ethylhexanol (2363.2 g) and tin(II)-2-ethylhexanoate (18.7 g). The mixture is heated to a set point temperature of 190° C. and maintained with stirring for a period of time, removing methanol via fractional distillation. Excess 2-ethylhexanol is removed by distillation under reduced pressure at 160° C. and then the reactor is cooled to 120° C.

Step 2: The Vigreux column is then removed from the reactor and tin(II)-2-ethylhexanoate (6.0 g) is added to the step 1 product (900.0 g), and the mixture is heated with stirring, to a set point temperature of 200° C. for a period of three hours. Excess 2-ethylhexanol is removed from the reactor contents by distillation under reduced pressure (20 mbar) and then the reactor is cooled to 120° C.

Step 3: Isobutyric anhydride (188.05 g) is added to the product of step 2 (754.02 g). The reactor is stirred at this temperature for 2 hours. Excess anhydride and acid formed during capping are removed under reduced pressure. Temperature is then increased to 160° C. and reduced pressure maintained for two hours. The reactor contents are then cooled to a set point temperature of 70° C. and NaHCO₃ aqueous solution (100 mL, 1 M) is added to the reactor with stirring. After stirring for 1 hour, water is removed under reduced pressure. Magnesium silicate (1% w/w), activated carbon (1% w/w) and MgSO₄ (1% w/w) are added to the reactor, then the material is filtered using a filter paper coated with 8% of magnesium silicate to yield the final product, which is a light yellow liquid.

Physical properties are tested for the products of Example 1 and Example 2, and results are shown in Table 1.

TABLE 1

| Properties | Example 1 | Example 2 |
|---|---|---|
| Viscosity at 40° C. (cSt) | 106 | 46.5 |
| Viscosity at 100° C. (cSt) | 16.3 | 8.83 |
| Viscosity Index | 167 | 173 |
| Pour Point (° C.) | −10 | −18 |
| Total Acid Number (mg | 0.41 | 0.26 |
| KOH/g) | | |
| Iodine Number (wt %) | <3 | <3 |
| Water (wt %) | 0.106 | 0.027 |
| % OH | 0.476 | 0 |
| OH # (mg KOH/g) | 15.7 | <3 |
| Color (Gardner) | 400 | 185 |
| Total Volatiles (ppm)¹ | 15 | 56 |
| Density at 20° C. (g/mL)² | 0.9099 | 0.9047 |

¹parts per million
²grams per milliliter

1. A process for preparing a double ester composition comprising the ordered steps of:
(1-a) at least partially homopolymerizing a hydroxylated fatty acid or fatty ester to form a fatty acid homopolymer;
(1-b) capping the fatty acid homopolymer with an acid, acid anhydride or ester to form a double ester; and
(1-c) transesterifying the fatty acid homopolymer with an alcohol to form a capped fatty acid homopolymer ester;
or
the ordered steps of
(2-a) transesterifying a hydroxylated fatty acid or fatty ester with an alcohol to form a hydroxylated fatty ester;
(2-b) homopolymerizing the hydroxylated fatty ester to form a fatty acid homopolymer ester; and
(2-c) capping the fatty acid homopolymer ester with an acid, acid anhydride or ester to form a double ester.

2. The process of claim 1 wherein
step (1-a) further includes using a tin-containing, titanium-containing, or nitrogen-containing catalyst, and forming as a second product an alcohol, and removing the formed alcohol;
step (1-b) further includes using, an acid that contains from 2 to 12 carbon atoms, an ester that contains from 3 to 13 carbon atoms, or an acid anhydride that contains from 4 to 24 carbon atoms, and also using a tin-containing, titanium-containing, or nitrogen-containing catalyst, and optionally recovering the double ester from an excess of the acid, the acid anhydride or the ester added in step (1-b);
step (1-c) further includes using a tin-containing, titanium-containing, or nitrogen-containing catalyst and optionally recovering the capped fatty acid homopolymer ester from the formed alcohol of step (1-a), or from residual alcohol added in step (1-c), or from an acid formed from a reaction of the capped fatty acid homopolymer ester with the acid, acid anhydride or ester added in step (1-b);
step (2-a) further includes using a tin-containing, titanium-containing, or nitrogen-containing catalyst and optionally recovering the hydroxylated fatty ester from residual or formed alcohol;
step (2-b) further includes using a tin-containing, titanium-containing or nitrogen-containing catalyst and removing the formed alcohol, optionally by using one or more of an entrainer, reduced pressure and nitrogen sparging; and
step (2-c) further optionally includes recovering the fatty acid homopolymer ester from an excess of the acid, acid anhydride or ester added as a reactant in step (2-b) and alcohol added as a reactant in step (2-c).

3. A process for preparing a double ester of a secondary hydroxy fatty acid or fatty ester, the process comprising either the ordered steps of (1-a) through (1-c), or of (2-a) through (2-c), the ordered steps being:
(1-a) partially homopolymerizing a hydroxylated fatty acid compound, using a tin-containing, titanium-containing or nitrogen-containing catalyst and removing formed alcohol, optionally by using one or more of an entrainer, reduced pressure and nitrogen sparging, to yield a product 1-X with distribution of compounds represented by Formula 1: 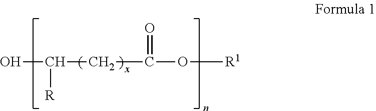
wherein in individual compounds R is an alkyl group that contains from 6 to 12 carbon atoms, R¹ is hydrogen or a methyl radical, x is an integer within a range of from 8 to 12 and n is an integer between 1 and 20, and the formed alcohol having the formula R¹OH;
(1-a1) optionally recovering product 1-X from residual R¹OH and, when used, the entrainer;
(1-b) reacting product 1-X with an acid that contains from 2 to 12 carbon atoms, an ester that contains from 3 to 13 carbon atoms, or an acid anhydride that contains from 4 to 24 carbon atoms, optionally using an additional amount of a tin-containing, titanium-containing or nitrogen-containing catalyst, and removing formed alcohol to yield a product 1-Y with a distribution of compounds represented by Formula 2: 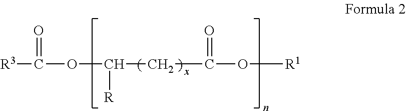
wherein R, R¹, x and n are as defined above and R³ is an alkyl group that contains from 1 to 11 carbon atoms;
(1-b1) optionally recovering product 1-Y from excess acid, acid anhydride or ester added as a reactant in step (1-b);
and
(1-c) reacting product 1-Y with an alcohol to form product 1-Z with a distribution of compounds represented by Formula 3: 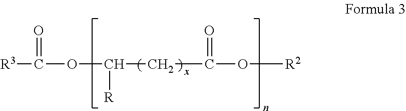
wherein R, R³, x and n are as defined above, R² an alkyl group that contains from 1 to 20 carbon atoms;
(1-c2) optionally recovering product 1-Z from alcohol and residual R¹OH added during (1-c) and acid formed during reaction of 1-Y with the acid, acid anhydride or ester added in (1-b);
or the ordered steps of:
(2-a) reacting a secondary hydroxyl fatty acid or fatty ester with an alcohol to form product 2-X with a distribution of compounds represented by Formula 4: 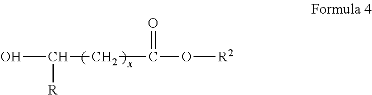
wherein R is an alkyl group that contains from 6 to 12 carbon atoms; R² is an alkyl group that contains from 1 to 20 carbon atoms, x is an integer within a range of from 8 to 12;
(2-a1) optionally recovering product 2-X from residual or formed R²OH;
(2-b) partially homopolymerizing product 2-X, using a tin-containing, titanium-containing or nitrogen-containing catalyst and removing the formed R²OH, optionally by using one or more of an entrainer, reduced pressure and nitrogen sparging, to yield a product 2-Y with distribution of compounds represented by Formula 5: 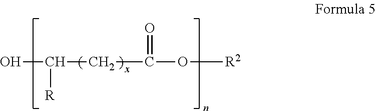
wherein in individual compounds R, R², and x are as defined above and n is an integer between 1 and 20;
(2-b1) optionally recovering product 2-Y from residual R²OH and, when used, the entrainer;
and
(2-c) reacting product 2-Y with an acid that contains from 2 to 12 carbon atoms, an ester that contains from 3 to 13 carbon atoms, or an acid anhydride that contains from 4 to 24 carbon atoms, optionally using an additional amount of a tin-containing, titanium-containing or nitrogen-containing catalyst, to yield a product 2-Z with distribution of compounds represented by Formula 6: 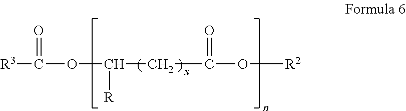
wherein R, R², R³, x and n are as defined above; and
(2-c1) optionally recovering product 2-Y from excess acid, acid anhydride or ester added as a reactant in (2-b) and alcohol added as a reactant in (2-c).

4. A double ester composition prepared by the process of claim 1.

5. The double ester composition of claim 4 wherein the double ester composition exhibits properties including at least one of a pour point that is less than or equal to −10° C. (measured according to ASTM D97); a viscosity index that is greater than or equal to 150; a kinematic viscosity at 40° C. that is more than 25 centistokes (cSt) (measured according to ASTM D445); a total acid number that is less than 1 milligram of potassium hydroxide per gram (mg KOH/g); a hydroxyl number that is less than or equal to 10; an iodine number that is less than 3 weight percent; and a renewable carbon level that is at least 50 percent by weight (measured according to ASTM 6866-08).

6. The double ester composition of claim 5 wherein the pour point is less than −15° C.; the kinematic viscosity at 40° C. is greater than 35 cSt; the total acid number is less than 0.5 mg KOH/g; and the hydroxyl number is less than 5.
